(19) United States
(12) Patent Application Publication
(10) Pub. No.: US 2020/0148996 A1
(43) Pub. Date: May 14, 2020

(54) CELL LYSIS AND BIOMOLECULE DISASSOCIATION SYSTEM AND METHOD FOR MASS SPECTROMETRY OR OTHER ANALYSIS

(71) Applicant: Covaris, Inc., Woburn, MA (US)

(72) Inventors: James A. LAUGHARN, JR., Boston, MA (US); William Murray SKEA, Saco, ME (US); Hans-Ulrich THOMANN, Stow, MA (US); Todd Anthony BASQUE, West Newbury, MA (US)

(73) Assignee: Covaris, Inc., Woburn, MA (US)

(21) Appl. No.: 16/667,086

(22) Filed: Oct. 29, 2019

Related U.S. Application Data
(60) Provisional application No. 62/757,811, filed on Nov. 9, 2018.

(51) Int. Cl. C12N 1/06; G01N 1/42; C12N 13/00; B01L 3/00

(52) U.S. Cl. CPC C12N 1/06; G01N 1/42; C12N 13/00; B01L 2300/1805; B01L 2200/0647; B01L 2300/1894; B01L 3/502

(57) ABSTRACT

Apparatus and method for lysing and recovering released material, such as proteins or other biomolecules. Sample material including cells may be at least partially frozen, thawed, cells lysed and biomolecules recovered in a single conduit, e.g., as the sample flows through the conduit. The conduit may include different zones for sample treatment, including a freezing zone, thawing zone, lysing/disassociation zone, etc., and protein or other recovered material may be delivered directly from the conduit to analysis equipment, such as a mass spectrometer. This allows a flow-through type processing of cells that avoids handling and transfer of sample material between different sample holders.

### RELATED APPLICATION

This Application claims priority under 35 U.S.C. § 119(e) to U.S. Provisional Application Ser. No. 62/757811, entitled “CELL LYSIS AND BIOMOLECULE DISASSOCIATION SYSTEM AND METHOD FOR MASS SPECTROMETRY OR OTHER ANALYSIS” filed Nov. 9, 2018, which is herein incorporated by reference in its entirety.

### BACKGROUND

### 1. Field of Invention

Methods and apparatus for lysing cells to release biomolecules and/or for disassociating biomolecules from the cells, e.g., to enable downstream analysis of proteins and peptides by mass spectrometry, or analysis of nucleic acids using PCR, sequencing or microarray hybridization, or other techniques.

### 2. Related Art

It is known that freezing of cells can lyse cell membranes, and thus release cell contents from the cell membrane, e.g., from U.S. Pat. No. 9,012,137.

### SUMMARY OF INVENTION

Aspects of the invention provide systems and methods for lysing cells and/or disassociating biomolecules from other cellular material while the cells move through a conduit. Thus, in some embodiments, cells can be introduced into an inlet of a conduit, move through the conduit where the cells are lysed and/or biomolecules are disassociated from other cell material, and disassociated biomolecules may exit an outlet of the conduit, e.g., for further analysis such as mass spectrometry, polymerase chain reaction (PCR), sequencing, etc., depending on the desired biomolecule to be analyzed. As an example, proteins and peptides recovered from lysed cells can be subjected to fingerprinting with or without prior hydrolysis by proteases, such as trypsin and the like. Released proteins can also be subjected to an ELISA-like assay in which epitopes are bound to immobilized antibodies (e.g., on a bead matrix) and in turn be quantitated by a sandwiched labeled antibody (e.g., fluorescently labeled). Released nucleic acids can be subjected to amplification (e.g., digital PCR, PCR or RT-PCR) to enable copy number variation (CNV) analysis. Another analysis may be by hybrid-capture following release and analysis by sequencing, PCR or the like. Since the methods and apparatus described herein enable isolation of biomolecules under conditions that preserve integrity of fragile analytes, quantitation of highly instable mRNAs such as those involved in the inflammatory (cytokine) pathways can be performed. (Schoenberg, D. R. & Maquat, L. E. Regulation of cytoplasmic mRNA decay. Nat. Rev. Genet. 13, 246-259 (2012).) In short, biomolecules recovered from cells can be subjected to any suitable analysis or subsequent treatment.

Some embodiments provide a flow-through type process where cells may be provided continuously or approximately continuously to the inlet of the conduit, and disassociated or otherwise released biomolecules from the cells, such as proteins or peptides separated from proteins, may exit the outlet of the conduit ready for analysis. As an example, cells may be provided to the inlet of the conduit via a flow cytometry or fluorescence-activated cell sorting (FACS) system. In other embodiments, frozen samples including cells may be introduced into the inlet of the conduit and moved through the conduit for processing. This arrangement may provide substantial advantages, such as significantly higher throughput rate, as compared to processes in which cells are frozen and then thawed in a capillary or other conduit without movement of the cells through the conduit. In some embodiments, the inventive system or method may include a conduit with multiple processing zones where cells receive different treatment as the cells move through the conduit. For example, a conduit may include a freezing zone where heat is removed from the cells and/or liquid with which the cells are mixed so as to freeze portions of the cells and/or liquid to form a frozen sample. The frozen sample may be moved in the conduit to a thawing zone, where frozen portions of the sample are thawed. (In alternate embodiments, a frozen sample may be thawed in a same location in the conduit as where the sample was frozen, or a previously frozen sample may be introduced to the conduit and moved to a thawing zone.) The thawed sample may be moved to a disassociation zone where focused acoustic energy or other treatment is used to lyse and/or disassociate biomolecules from other material released from the cells. Freezing of the sample may lyse cells and/or disrupt cell membranes to make the cells more susceptible to lysing, or more easily lysed, by focused acoustic energy. As a result, lower energy acoustic energy may be employed for lysing and/or disassociation of biomolecules, which may help preserve biomolecules in an undisrupted and/or biologically active state as found in an unlysed cell. In some cases, focused acoustic energy may be used to aid in thawing of a frozen sample, and in such cases, the focused acoustic energy may be used to thaw the sample, lyse cells and disassociate biomolecules from other cellular material. This processing may occur in the conduit, and as a sample moves through the conduit, allowing multiple samples to be held and processed in the conduit in some embodiments. As discussed more below, the conduit may include additional sample treatment zones, such as a guard column or filter zone where cellular debris is separated from protein or other biomolecular material from the cells using a filtering or other separation process (such as a centrifugation zone where cell materials are separated using centrifugation), a reduction/alkylation chamber where separated protein material can be reduced and/or alkylated, a digestion chamber where protein material may be enzyme digested to form separated peptides from the protein material, and/or other processing zones. Proteins, peptides and/or other processed material output from the conduit may be directed to any suitable analysis or other treatment tool. For example, peptides may be directed to a liquid chromatography column to separate the peptide material for subsequent analysis, such as mass spectrometry. In other embodiments, material output from the conduit may be subjected to direct, rapid thermocycling (e.g., by putting output nucleic acids in a capillary system coupled to a Peltier element that is adapted to rapidly thermocycle a sample in a capillary). The capillary system may be loaded with immobilized nucleic acid capturing probes to capture specific nucleic acids by hybridization at typical temperatures, e.g., 45 to 70 degrees C. Accordingly, in some embodiments, the system and method may allow cells to be introduced at an inlet to the conduit, and separated protein and/or peptide material suitable for mass spectrometry or other analysis may exit at an outlet of the conduit. This processing may be done without human, robotic or other handling of cell samples after the cell samples are introduced into the conduit.

In one aspect of the invention, a method of collecting material from cells for analysis includes providing a sample including a plurality of whole cells and liquid into a conduit, and moving the sample in the conduit into a freezing zone where at least some of the liquid in the sample freezes so as to form a frozen sample. In some cases, the freezing may serve to lyse cell membranes, or to disrupt cell membranes to make them more susceptible by lysing. The frozen sample may be exposed to energy while the frozen sample is in the conduit to thaw the frozen sample and form a thawed sample, e.g., focused acoustic energy and/or thermal energy may be provided to the frozen sample to thaw frozen portions of the sample. The thawed sample may be exposed to focused acoustic energy while the thawed sample is in the conduit to create a focal zone of acoustic energy at the thawed sample to lyse the plurality of whole cells and release contents of the plurality of whole cells and/or to disassociate biomolecules from the plurality of whole cells. The thawed sample including the released contents of the plurality of whole cells may be moved in the conduit to a subsequent treatment area of the conduit, e.g., the released contents may be output to a subsequent analysis or other treatment process, such as PCR, mass spectrometry, nucleic acid hybridization, etc.

In some embodiments, moving the sample in the conduit into a freezing zone includes freezing contents inside of the plurality of cells. Thus, the entire contents of cells may be frozen (at least to the extent reasonably possible or useful) as well as liquid material around the cells. Alternately, only liquid around the cells may be frozen, or interior portions of cells may be frozen. In some cases, freezing may disrupt cell membranes of the plurality of cells by freezing portions of the sample, e.g., at least some cell membranes of the plurality of cells may be lysed by freezing portions of the sample.

Exposing the frozen sample to energy while the frozen sample is in the conduit to thaw the frozen sample may in some cases include exposing the frozen sample to focused acoustic energy and/or heat energy (radiative and/or conductive) to thaw the frozen sample.

Moving the thawed sample to a subsequent treatment area of the conduit may include a variety of different techniques, including outputting the thawed sample from the conduit. In some cases, such as where proteins or peptides in the released contents are to be analyzed, released contents of cells may be moved into a separation chamber to separate protein material in the released contents from other material in the released contents. The separation chamber may include a guard column or filter to separate protein material from other material in the released contents. The separated protein material may then be moved in the conduit to a reduction/alkylation chamber in which the separated protein material is reduced and/or alkylated to form reduced/alkylated protein material. This process may include exposing the separated protein to focused acoustic energy in the reduction/alkylation chamber to mix the separated protein with reduction/alkylation reagents in the reduction/alkylation chamber. Thereafter, reduced/alkylated protein material may be moved in the conduit to a digestion chamber in which the reduced/alkylated protein material is digested by an enzyme to produce a plurality of separated peptides. The plurality of separated peptides may then be moved in the conduit to a liquid chromatography column to deliver the plurality of separated peptides to a mass spectrometry device. Of course, other treatment processes may be performed on cell material in a conduit, including PCR, nucleic hybridization, etc., as aspects of the invention are not necessarily limited in this regard.

In another aspect of the invention, a system for collecting material from cells for analysis includes a conduit extending from an inlet to an outlet and adapted to receive a sample including a plurality of whole cells and a liquid at the inlet. In some cases, the conduit may receive the sample in liquid form, or in frozen form. A pump may be arranged to move the (frozen or liquid) sample in the conduit from the inlet toward the outlet of the conduit, e.g., using a fluid pump or other moving mechanism such as an auger or piston. The conduit may include a freezing zone adapted to freeze at least a portion of the sample while the sample remains in the conduit. Where the sample is introduced into the conduit in frozen form, the frozen sample may be further cooled if desired. A heat transfer device may be adapted to transfer heat from the sample at the freezing zone so as to freeze at least a portion of the sample. In other arrangements, the conduit need not include a freezing zone or heat transfer device to freeze a sample. The conduit may also include a thawing zone adapted to deliver energy to a frozen sample in the conduit to thaw the sample, and a focused acoustic energy apparatus adapted to transmit focused acoustic energy toward the sample while the sample is in the conduit to lyse the plurality of whole cells and/or to disassociate biomolecules from the plurality of whole cells.

In some embodiments, the freezing zone and the thawing zone are located in a same portion of the conduit, or the two zones may be separate, e.g., with the freezing zone located nearer the inlet of the conduit than the thawing zone. The pump may be arranged to move a frozen sample in the conduit from the freezing zone to the thawing zone, e.g., a magnetically driven piston in the conduit may move a frozen sample slug in the conduit. In some cases, the acoustic energy apparatus may be adapted to expose a frozen sample in the conduit to focused acoustic energy to thaw the frozen sample. Thus, the acoustic energy apparatus may perform both thawing and lysing/disassociation functions, or may include two acoustic transducers to perform the two functions. Alternately, the system may include a different type of heat transfer device to transfer heat to a frozen sample, such as a Peltier device, a refrigeration circuit, an electrical resistance heater, etc.

In another aspect of the invention, a method of collecting material from cells for analysis includes providing a frozen sample including a plurality of whole cells and at least partially frozen liquid into a conduit, and moving the frozen sample in the conduit. The frozen sample may be exposed to energy while the frozen sample is in the conduit to thaw the frozen sample and form a thawed sample, and the thawed sample may be exposed to focused acoustic energy while the thawed sample is in the conduit to create a focal zone of acoustic energy at the thawed sample to lyse the plurality of whole cells and release contents of the plurality of whole cells and/or to disassociate biomolecules from the plurality of whole cells. The thawed sample including the released contents of the plurality of whole cells may be moved in the conduit to a subsequent treatment area of the conduit, such as a section to perform filtering or other separation of cell contents, processing of proteins, PCR of nucleic acids, etc.

In some embodiments, moving the frozen sample in the conduit includes moving the frozen sample to a thawing zone of the conduit, and the frozen sample may be exposed to focused acoustic energy and/or heat energy to thaw the frozen sample.

Other advantages and novel features of the invention will become apparent from the following detailed description of various non-limiting embodiments when considered in conjunction with the accompanying figures and claims.

### BRIEF DESCRIPTION OF THE DRAWINGS

Aspects of the invention are described with reference to the following drawings in which numerals reference like elements, and wherein:

FIG. 1 is a schematic diagram of a cell processing system in an illustrative embodiment.

### DETAILED DESCRIPTION

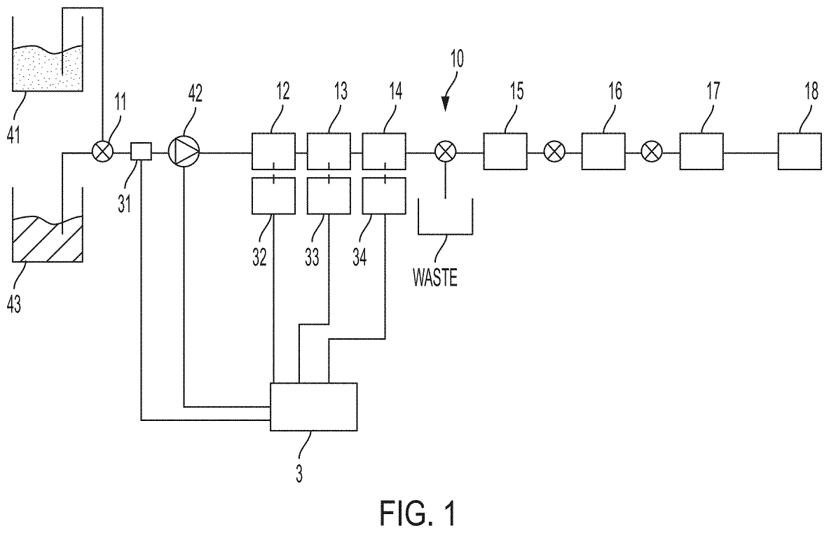

FIG. 1 shows a schematic diagram of a system arranged to process cells mixed with a liquid. The sample mixture may include a plurality of only one type of cell, such as a plurality of the same type of yeast cells, or may include cells of two or more different types, such as blood cells and bacteria. Some sample mixtures having two or more different cell types may be processed by selectively lysing cells of one type while leaving cells of another type to be isolated in whole form. This may allow for easier separating of the whole cells because the lysed cells are no longer in whole form. In some embodiments, a flow cytometry or fluorescence-activated cell sorting (FACS) may be employed to provide desired cells or groups of cell types as an input to the system.

The illustrative embodiment of FIG. 1 is just that—an illustrative embodiment and it should be understood that various modifications of the system may be made. In this embodiment, a sample material including a liquid and whole cells is held in a reservoir or tank 41 which may be of any suitable size, shape or configuration. For example, the tank 41 may contain less than a liter of mixture, or 10's or 100's of liters, may include a bulk stirring apparatus (such as a mixer) to keep cells suitably suspended or otherwise agitated, a heater or chiller to heat or cool the mixture, and so on. The cells may be arranged in any concentration. Note also that the liquid the cells are combined with in the tank 41 could be a liquid added to a group of cells, e.g., water mixed with a group of yeast cells, or may be included with the cells as harvested or otherwise collected, e.g., the tank 41 may include a blood sample that includes blood cells and liquid in the form of blood serum or plasma. In this context, the tank 41 need not have a large volume, and may include a blood collection tube (BCT), syringe, or other vessel.

The cells may be introduced into a conduit 10 via a valve 11, and any number or other amount of cells may be selectively introduced into the conduit 10, e.g., 500 cells may be introduced into the conduit 10 via the valve 11. Cells may be introduced into the conduit 10 by opening the valve 11 for a period of time and/or otherwise to allow a desired number of cells to flow into the conduit 10. The valve 11 may define an inlet for the conduit 10, or another portion of the system may define the inlet for the conduit 10 depending on the system arrangement. For example, the valve 11 may include a septum through which a volume of cells can be injected into the conduit 10 by syringe, a pump, or other technique. In another embodiment, the tank 41 may include a recirculation line and pump that provides cells and liquid under pressure at the valve 11 so that when the valve 11 is opened, cells may be injected into the conduit 10, but when the valve 11 is closed, the cells recirculate to the tank 41. In other embodiments, cells may be provided to the inlet of the conduit 10 as part of a frozen or partially frozen sample. For example, slugs of frozen or partially frozen material containing cells may be introduced to the inlet of the conduit 10, e.g., by opening the valve 11 to allow a sample slug entry into the conduit 10. In other arrangements, frozen sample material may be introduced into the conduit 10 by hand, a robotic loading machine, a conveyor or other delivery device.

Sample material in the conduit 10 may be moved by a pump 42, such as a peristaltic pump or any other suitable fluid or solid moving mechanism, such as an auger or piston where sample material is frozen within or introduced to the conduit 10 in frozen form. In some cases, the pump 42 may function to draw cells into the conduit 10, e.g., relative low pressure at the valve 11 created by the pump 42 may draw cells into the conduit 10. The pump 42 may be selected for its capability to move cells in the sample mixture without damaging the cells, if desired. The pump 42 may be operated under the control of a control circuit 3 which may include various sensors, input/output devices, actuators, etc. as discussed in more detail below. For example, an input detector 31 may detect a cell concentration and/or number of cells entering the conduit 10, e.g., by optical or other turbidity measure of the sample mixture. Information from the detector 31 may be used to measure and control operation of the system, at least in part, such as control of the valve 11 to control a number or volume of cells introduced into the conduit 10. The system may also include a liquid tank 43 that holds a liquid to be mixed with cells introduced into the conduit 10 and/or liquid used between discrete sample slugs of cells. For example, a volume of cells may be introduced into the conduit 10 at the valve 11, and then a volume of liquid from the tank 43 may be introduced into the conduit 10. Thereafter, another volume of cells may be introduced into the conduit 10, followed by another volume of liquid. This alternate introduction of cells and liquid may be accomplished by suitable operation of the valve 11, and may function to separate discrete cell samples (sample slugs) in the conduit 10 from each other. In some cases, the liquid may be a glycerin or other material that is resistant to mixing with the cell sample material and may help keep sample slugs separated from each other. In other arrangements, air or other gas may be used to separate cell samples from each other instead of a liquid material. This may allow for the processing of individual sample slugs so that recovered biomolecules from one sample can be kept, and analyzed, separate from another. In other arrangements, liquid from the tank 43 may be mixed with the cells introduced from the tank 41, e.g., to dilute the cells or to combine a desired reagent or buffer with the cells, and sample material flowing in the conduit 10 may have cells evenly distributed along the conduit path. Of course, the tank 43 can be eliminated and cells combined with desired reagent, buffer or other material in the tank 41. In some cases, sample material containing cells may flow continuously, or nearly continuously, in the conduit 10. Cells may flow in the conduit at a rate of about 0.2 microliters per minute to 500 microliters per minute in some embodiments.

A sample containing cells (whether arranged as a discrete frozen or liquid slug, or as a continuous flow in the conduit 10) is moved by the pump 42 into a freezing zone 12 of the conduit 10 so that heat can be removed from the sample to freeze at least a portion of the liquid and/or the cells in the sample. Where samples are introduced into the conduit 10 in frozen form, the samples may be cooled further in the freezing zone 12 (e.g., to more completely freeze cells in the sample), or if further freezing is not necessary, the sample may bypass the freezing zone 12. (In some cases, the freezing zone 12 may be eliminated entirely.) A heat transfer device 32, which may include a Peltier device, a cryo-cooling system of single or dual stage configuration (e.g., including a liquid nitrogen flow path), a refrigerant system, or other arrangement to remove heat from the cell sample. As an example, liquid nitrogen may contact the exterior of the conduit 10 at the freezing zone 12, which removes heat from the conduit 10, and thus from the cell sample, so as to freeze at least a portion of the sample. The heat transfer device 32 may operate under the control of the control circuit 3, and may include temperatures sensors and/or other devices so that feedback control of the heat transfer device 32 can be employed, e.g., so the sample is frozen to a desired extent and/or for a desired period of time. Freezing of at least a portion of the cell sample may lyse cells and/or otherwise disrupt cell membranes of the cells so as to make the cells more easily lysed by other processes. In some embodiments, cells themselves need not be frozen, or completely frozen. Instead, only a portion of the sample material may be frozen, and the partial freezing may be sufficient to disrupt cell membranes to make them more susceptible to lysing in a later process.

A frozen sample may be moved to a thawing zone 13 of the conduit 10 so that the sample may be at least partially thawed. The pump 42 may move a frozen sample in the conduit 10 from the freezing zone 12 to the thawing zone 13 using suitable pressure or other motive force, e.g., a turning auger or piston may turn to push the sample into the thawing zone. In some cases, at least an outermost layer of the frozen sample may be thawed in the freezing zone 12 to aid in moving the frozen sample to the thawing zone 13. That is, freezing of the sample may cause the sample to “stick” to the conduit 10 inner wall at the freezing zone 12, and thawing of a relatively thin layer of the sample in the freezing zone 12 may aid in moving the sample from the freezing zone 12 to the thawing zone 13. Thawing of a portion of the sample in the freezing zone 12 may be done by reversing operation of the heat transfer device 32, e.g., the Peltier device or refrigerant circuit may be operated to transfer heat into the sample, or a separate device may be employed, such as a focused acoustic energy device. In some cases, the freezing zone 12 and the thawing zone 13 may be located in a same place in a conduit 10, e.g., a sample may be frozen and then thawed at a same location of the conduit 10.

Once moved to the thawing zone 13, a heat transfer device 33 may be used to transfer heat energy into the sample to thaw frozen portions. The heat transfer device 33 may operate under the control of the control circuit 3 and may include temperature sensors and/or other devices to enable feedback control. The heat transfer device 33 may include a Peltier device, a refrigerant circuit, a flowpath for heated liquid or other material, an acoustic energy device, etc. In some embodiments, the heat transfer device 33 may receive heat removed from the conduit 10 and sample by the heat transfer device 32, and introduce at least a portion of that heat back into the sample. For example, a “cold” portion of a Peltier device may operate to freeze a sample at the freezing zone 12, and a “hot” portion of the same Peltier device may operate to thaw a sample at the thawing zone 13. Thus, a single Peltier device may operate as part of both heat transfer devices 32, 33.

The thawed sample may next be moved to a lysing/disassociation zone 14 where the thawed sample may be lysed and/or biomolecules released from lysed cells may be disassociated from other material released from the cells. The system may include a focused acoustic energy apparatus 34 that operates to expose the thawed sample, while in the conduit 10, to focused acoustic energy at a focal zone so as to lyse cells and release cellular material and/or disassociate biomolecules such as proteins from other cellular material. U.S. Pat. No. 9,080,167 discusses focused acoustic energy systems suitable for such lysing and disassociation purposes and is incorporated by reference herein for its disclosure of focused acoustic systems. In addition, Covaris, Inc. of Woburn, Mass. manufactures and sells focused acoustic energy systems suitable for use in all focused acoustic applications described herein. In some embodiments, the focused acoustic energy may also be used to thaw a frozen sample, and thus the thawing zone 13 and the lysing/disassociation zone 14 may be located at the same place along the conduit 10, and the focused acoustic energy system may form all or part of the heat transfer device 33. In some cases, the focused acoustic energy system may be operated in two modes, i.e., one mode for thawing the sample, and another mode for lysing/disassociation. At the output of the lysing/disassociation zone 14, disassociated biomolecules released from the cells will be combined with other cellular material, including cell membrane material, etc. This output material could be put to further processing and/or analysis, as is known in the art.

In this illustrative embodiment, the conduit 10 includes additional processing components that operate on the disassociated biomolecules output at the lysing/disassociation zone 14. In this embodiment, the conduit 10 further includes a guard column or filter 15 that operates to separate recovered proteins from other cellular material released by the lysed cells. That is, recovered proteins pass through the guard column/filter 15 (e.g., under the motive force of the pump 42 or other mover) and are collected in a reduction/alkylation chamber 16 while other cellular material is collected in the guard column/filter 15. Cellular material collected in the guard column/filter 15 may be backwashed or otherwise removed from the guard column/filter 15 by operating suitable valves (shown schematically in FIG. 1) and reversing flow in the guard column/filter 15 so the cellular material is dumped to a waste line or other collection area. Such backwashing may be performed for each sample where samples are provided in discrete form, or after a run of sample material of a particular volume or time. In the reduction/alkylation chamber 16, the recovered proteins may be mixed with suitable reagents or other materials to reduce and/or alkylate the proteins. Although not shown, a focused acoustic energy system may be used to mix the proteins with the reagents or other material in the reduction/alkylation chamber 16. The reduced/alkylated protein material may be delivered to a digestion chamber 17 where the protein material is digested by an enzyme, such as trypsin, to produce individual peptides. Again, a focused acoustic energy system may be used to mix or otherwise treat materials in the digestion chamber 17, and the trypsin or other enzyme may be immobilized. The peptides may be next delivered to a liquid chromatography device 18 to separate the peptides for subsequent analysis, such as by a mass spectrometry device (not shown). As a result, whole cells may be introduced to an input of the conduit 10, and the cells may be processed within the conduit 10 to ultimately deliver released proteins, peptides or other recovered material from lysed cells for analysis without the sample material exiting the conduit 10. This can avoid time-consuming and potentially contaminating human or other handling, transfer of sample material between multiple sample holders, etc.

As discussed above, other treatment or analysis processes may be employed for use with biomolecules output from the lysing/disassociation zone 14 than those shown in FIG. 1. As an example, a qPCR-based analysis of isolated DNA or RNA may be employed. In this illustrative case, cell lysate is passed into the guard column/filter 15 where it is mixed with a binding buffer that enables the DNA to bind to a glass/silica filter at the end of the guard column/filter 15. A reagent line and a waste line may be connected to the input and output of the guard column/filter 15, respectively, e.g., coupled to valves. After the binding buffer is mixed with the cell lysate, e.g., by applying focused acoustic energy to contents of the guard column/filter 15, non-bound contaminants in the guard column/filter 15 may be washed into the waste line. After adding a wash solution through the reagent inlet, the bound DNA may be washed and the DNA eluted into a next chamber, where the DNA is mixed with PCR or RCA reagents (buffer, nucleotides, enzymes and fluorescent intercalating dye), and then moved to a next chamber where the DNA is amplified. Amplification may be done using a Peltier element coupled to the amplification chamber to perform needed thermocycling. PCR products may then be analyzed fluorescently by endpoint. Quantitation can be achieved by continuous monitoring of the fluorescence of a small aliquot of the reaction (e.g., 1/150 to 1/100 of the entire volume) that is released into an optical detection chamber.

Yet another configuration includes the quantitative analysis of mRNA species such as those involved in the cytokine response pathway. Such mRNAs are regulated post-transcriptionally thereby shortening their half-life (Anderson, P. Post-transcriptional control of cytokine production. Nat. Immunol. 9, 353-359 (2008)). Thus, fast and reproducible lysis of the cells and extraction of mRNA is crucial for reliable quantitative analysis. To perform this processing, the conduit 10 would be modified so that the cell lysate containing the desired mRNAs from the lysing/disassociation zone 14 is first mixed with DNAse to deplete any co-released DNA in a depletion chamber downstream of the lysing/disassociation zone 14. The material may then be moved from the depletion chamber to a next hybridization chamber and mixed with a mixture of immobilized capture probes (e.g., on beads) and a hybridization buffer. The probes can be oligo-T (to capture mRNA by hybridization against poly A tails) or specific probes complementary to sequences of mRNA species of interest. Hybridization is being done in presence of pulsed focused acoustic energy and at temperatures of 45-70 degrees C. After washing non-bound nucleic acids away (beads can simply be held back in the hybridization chamber by a coarse filter), the mRNA captured are released by heating to 95 degrees C. for 2-3 seconds and washed into a next chamber, were the mRNA are mixed with RT-PCR reagents, preferably those allowing one-tube, one-step RT-PCR (e.g., Roche Tth), and quantified as described above.

Another configuration that will take advantage of the preservation nature of the system, i.e., freezing cells to preserve the biological/chemical stage of the biomolecules contained therein, is the simultaneous analysis of mRNA (transcriptome) and chromatin (epigenetic pattern) such as described in Cao et al. (2018) and Clark et al. (2017) (Cao, J., et al. 2018. Joint profiling of chromatin accessibility and gene expression in thousands of single cells. Science 10.1126/science.aau0730; Clark, S., et al. 2017, scNMT-seq enables joint profiling of chromatin accessibility DNA methylation and transcription in single cells. Nature Communications DOI: 10.1038/s41467-018-03149-4). In these assays, RNA is isolated and subsequently analyzed by either sequencing, qPCR and or hybrid-capture followed by sequencing. In parallel, open (non-histone wrapped) chromatin is methylated and subsequently bi-sulfite sequenced. The system described herein is suited to perform the necessary steps, such as 1. Diluting the cells to low concentrations, 2. Freezing them to preserve mRNA integrity and epigenetic patterns, 3. Lysing the cells with AFA to release mRNA and separating it from the cell debris and nuclei containing the chromatin, 4. Treating the chromatin with a CpG methyltransferase, 5. Homogenizing and releasing the DNA with AFA, and 6. Releasing the RNA and DNA that were isolated in parallel in separated conduits (after step 3.) for sequencing from the system.

Regarding focused acoustic energy systems, under the control of the control circuit 3, an acoustic transducer may produce acoustic energy within a frequency range of between about 100 kilohertz and about 100 megahertz such that a focal zone has a width of about 2 centimeters or less. The focal zone of the acoustic energy may be any suitable shape, such as spherical, ellipsoidal, rod-shaped, or column-shaped, for example, and be positioned suitably relative to the conduit 10 and sample. The focal zone may be larger than the sample volume, or may be smaller than the chamber volume. U.S. Pat. Nos. 6,948,843 and 6,719,449 are incorporated by reference herein for details regarding the construction and operation of an acoustic transducer and its control. The focal zone may be stationary relative to the sample, or it may move relative to the sample.

In some embodiments, the transducer can be formed of a piezoelectric material, such as a piezoelectric ceramic. The ceramic may be fabricated as a “dome,” which tends to focus the energy. One application of such materials is in sound reproduction; however, as used herein, the frequency is generally much higher and the piezoelectric material would be typically overdriven, that is driven by a voltage beyond the linear region of mechanical response to voltage change, to sharpen the pulses. Typically, these domes have a longer focal length than that found in lithotriptic systems, for example, about 20 cm versus about 10 cm focal length. Ceramic domes can be damped to prevent ringing or undamped to increase power output. The response may be linear if not overdriven. The high-energy focus zone of one of these domes is typically cigar-shaped. At 1 MHz, the focal zone is about 6 cm long and about 2 cm wide for a 20 cm dome, or about 15 mm long and about 3 mm wide for a 10 cm dome. The peak positive pressure obtained from such systems at the focal zone 21 is about 1 MPa (mega Pascal) to about 10 MPa pressure, or about 150 PSI (pounds per square inch) to about 1500 PSI, depending on the driving voltage. he focal zone, defined as having an acoustic intensity within about 6 dB of the peak acoustic intensity, is formed around the geometric focal point. It is also possible to generate a line-shaped focal zone, e.g., that spans the length of the chamber as discussed above.

To control an acoustic transducer, the system control circuit 3 may provide control signals to a load current control circuit, which controls a load current in a winding of a transformer. Based on the load current, the transformer may output a drive signal to a matching network, which is coupled to the acoustic transducer and provides suitable signals for the transducer to produce desired acoustic energy. Moreover, the system control circuit 3 may control various other system functions, such as receiving operator input (such as commands for system operation by employing a user interface), outputting information (e.g., to a visible display screen, indicator lights, sample treatment status information in electronic data form, and so on), and others. Thus, the system control circuit 3 may include any suitable components to perform desired control, communication and/or other functions. For example, the system control circuit 3 may include one or more general purpose computers, a network of computers, one or more microprocessors, etc. for performing data processing functions, one or more memories for storing data and/or operating instructions (e.g., including volatile and/or non-volatile memories such as optical disks and disk drives, semiconductor memory, magnetic tape or disk memories, and so on), communication buses or other communication devices for wired or wireless communication (e.g., including various wires, switches, connectors, Ethernet communication devices, WLAN communication devices, and so on), software or other computer-executable instructions (e.g., including instructions for carrying out functions related to controlling the load current control circuit as described above and other components), a power supply or other power source (such as a plug for mating with an electrical outlet, batteries, transformers, etc.), relays and/or other switching devices, mechanical linkages, one or more sensors or data input devices (such as a video camera or other imaging device to capture and analyze image information regarding the sample or other components, turbidity or other measurement of cell concentrations in a flow into or out of the zones of the conduit 10, and so on), user data input devices (such as buttons, dials, knobs, a keyboard, a touch screen or other), information display devices (such as an LCD display, indicator lights, a printer, etc.), and/or other components for providing desired input/output and control functions. Also, the control circuit 3 may include one or more components to detect and control a temperature of a coupling medium such as a refrigeration system to chill the coupling medium, a degassing system to remove dissolved gas from the coupling medium, etc.

Although not necessarily critical to employing aspects of the invention, in some embodiments, sample treatment control may include a feedback loop for regulating at least one of acoustic energy location, frequency, pattern, intensity, duration, and/or absorbed dose of the acoustic energy to achieve the desired result of acoustic treatment. One or more sensors may be employed by the control circuit 3 to sense parameters of the acoustic energy emitted by the transducer and/or of the mixture, and the control circuit 3 may adjust parameters of the acoustic energy and/or of the mixture (such as flow rate, concentration, etc.) accordingly. Thus, control of the acoustic energy source may be performed by a system control unit using a feedback control mechanism so that any of accuracy, reproducibility, speed of processing, control of temperature, provision of uniformity of exposure to sonic pulses, sensing of degree of completion of processing, monitoring of cavitation, and control of beam properties (including intensity, frequency, degree of focusing, wave train pattern, and position), can enhance performance of the treatment system. A variety of sensors or sensed properties may be used by the control circuit for providing input for feedback control. These properties can include sensing of temperature, cell concentration or other characteristic of the mixture; sonic beam intensity; pressure; coupling medium properties including temperature, salinity, and polarity; chamber position; conductivity, impedance, inductance, and/or the magnetic equivalents of these properties, and optical or visual properties of the mixture. These optical properties, which may be detected by a sensor typically in the visible, IR, and UV ranges, may include apparent color, emission, absorption, fluorescence, phosphorescence, scattering, particle size, laser/Doppler fluid and particle velocities, and effective viscosity. Mixture integrity and/or comminution can be sensed with a pattern analysis of an optical signal from the sensor. Particle size, solubility level, physical uniformity and the form of particles could all be measured using instrumentation either fully standalone sampling of the fluid and providing a feedback signal, or integrated directly with the focused acoustical system via measurement interface points such as an optical window. Any sensed property or combination thereof can serve as input into a control system. The feedback can be used to control any output of the system, for example beam properties, flow in the chamber, treatment duration, and losses of energy at boundaries and in transit via reflection, dispersion, diffraction, absorption, dephasing and detuning.

The desired result of acoustic treatment, which may be achieved or enhanced by use of ultrasonic wavetrains, can be, without limitation, moving cells in the chamber to aid in separating cells from a mixture, but also heating the mixture, cooling the mixture, fluidizing the mixture, micronizing the mixture, mixing the mixture, stirring the mixture, disrupting the mixture, permeabilizing a component of the mixture, forming a nanoemulsion or nano formulation, enhancing a reaction in the mixture, solubilizing, sterilizing the mixture, lysing, extracting, comminuting, catalyzing, and/or selectively degrading at least a portion of a mixture. In embodiments specifically discussed herein, specialized mixing of the mixture is particularly effective in enhancing ligation reactions. Sonic waves may also enhance filtration, fluid flow in conduits, and fluidization of suspensions. Processes in accordance with the present disclosure may be synthetic, analytic, or simply facilitative of other processes such as stirring.

While aspects of the invention have been described with reference to various illustrative embodiments, such aspects are not limited to the embodiments described. Thus, it is evident that many alternatives, modifications, and variations of the embodiments described will be apparent to those skilled in the art. Accordingly, embodiments as set forth herein are intended to be illustrative, not limiting. Various changes may be made without departing from the spirit of aspects of the invention.

1. A method of collecting material from cells for analysis, comprising:
providing a sample including a plurality of whole cells and liquid into a conduit;
moving the sample in the conduit into a freezing zone where at least some of the liquid in the sample freezes so as to form a frozen sample;
exposing the frozen sample to energy while the frozen sample is in the conduit to thaw the frozen sample and form a thawed sample;
exposing the thawed sample to focused acoustic energy while the thawed sample is in the conduit to create a focal zone of acoustic energy at the thawed sample to lyse the plurality of whole cells and release contents of the plurality of whole cells and/or to disassociate biomolecules from the plurality of whole cells; and
moving the thawed sample including the released contents of the plurality of whole cells in the conduit to a subsequent treatment area of the conduit.

2. The method of claim 1, wherein the step of moving the sample in the conduit into a freezing zone includes freezing contents inside of the plurality of cells.

3. The method of claim 1, wherein the step of moving the sample in the conduit into a freezing zone includes disrupting cell membranes of the plurality of cells by freezing portions of the sample.

4. The method of claim 1, wherein the step of moving the sample in the conduit into a freezing zone includes lysing at least some cell membranes of the plurality of cells by freezing portions of the sample.

5. The method of claim 1, wherein the step of exposing the frozen sample to energy while the frozen sample is in the conduit to thaw the frozen sample includes exposing the frozen sample to focused acoustic energy to thaw the frozen sample.

6. The method of claim 1, wherein the step of exposing the frozen sample to energy while the frozen sample is in the conduit to thaw the frozen sample includes exposing the frozen sample to heat energy to thaw the frozen sample.

7. The method of claim 1, wherein the step of moving the thawed sample to a subsequent treatment area of the conduit includes moving the thawed sample into a separation chamber to separate protein material in the released contents from other material in the released contents.

8. The method of claim 7, wherein the separation chamber includes a guard column or filter to separate protein material from other material in the released contents.

9. The method of claim 7, further comprising moving the separated protein material in the conduit to a reduction/alkylation chamber in which the separated protein material is reduced and/or alkylated to form reduced/alkylated protein material.

10. The method of claim 9, wherein the step of moving the separated protein material in the conduit to the reduction/alkylation chamber includes exposing the separated protein to focused acoustic energy in the reduction/alkylation chamber to mix the separated protein with reduction/alkylation reagents in the reduction/alkylation chamber.

11. The method of claim 9, further comprising moving the reduced/alkylated protein material in the conduit to a digestion chamber in which the reduced/alkylated protein material is digested by an enzyme to produce a plurality of separated peptides.

12. The method of claim 11, further comprising moving the plurality of separated peptides in the conduit to a liquid chromatography column to deliver the plurality of separated peptides to a mass spectrometry device.

13. A system for collecting material from cells for analysis, comprising:
a conduit extending from an inlet to an outlet and adapted to receive a sample including a plurality of whole cells and a liquid at the inlet, the conduit including:
a pump to move the sample in the conduit from the inlet toward the outlet;
a freezing zone adapted to freeze at least a portion of the sample while the sample remains in the conduit; and
a thawing zone adapted to deliver energy to a frozen sample in the conduit to thaw the sample;
a heat transfer device arranged to transfer heat from the sample at the freezing zone so as to freeze at least a portion of the sample; and
a focused acoustic energy apparatus adapted to transmit focused acoustic energy toward the sample while the sample is in the conduit to lyse the plurality of whole cells and/or to disassociate biomolecules from the plurality of whole cells.

14. The system of claim 13, wherein the freezing zone and the thawing zone are located in a same portion of the conduit.

15. The system of claim 13, wherein the freezing zone is located nearer the inlet of the conduit than the thawing zone.

16. The system of claim 15, wherein the pump is arranged to move a frozen sample in the conduit from the freezing zone to the thawing zone.

17. The system of claim 13, wherein the focused acoustic energy apparatus is adapted to expose a frozen sample in the conduit to focused acoustic energy to thaw the frozen sample.

18. The system of claim 13, wherein the heat transfer device includes a liquid nitrogen chiller adapted to remove heat from the sample in the conduit at the freezing zone.

19. The system of claim 13, wherein the heat transfer device includes a Peltier device adapted to remove heat from the sample in the conduit at the freezing zone.

20. The system of claim 13, wherein the conduit further comprises a separation chamber adapted to separate protein material from other material released from the lysed cells.

21. The system of claim 20, wherein the separation chamber includes a guard column or filter to separate the protein material from the other material.

22. The system of claim 20, wherein the conduit further comprises a reduction/alkylation chamber adapted to reduce and/or alkylate the separated protein material to form reduced/alkylated protein material.

23. The system of claim 22, further comprising a focused acoustic energy source adapted to expose the separated protein to focused acoustic energy in the reduction/alkylation chamber to mix the separated protein with reduction/alkylation reagents in the reduction/alkylation chamber.

24. The system of claim 22, wherein the conduit further comprises a digestion chamber into which the reduced/alkylated protein material is moved, and in which the reduced/alkylated protein material is digested by an enzyme to produce a plurality of separated peptides.

25. The system of claim 24, wherein the conduit further comprises a liquid chromatography column adapted to process the plurality of separated peptides for further analysis.

26. The system of claim 25, wherein the further analysis includes mass spectrometry of the plurality of separated peptides.

27. A method of collecting material from cells for analysis, comprising:
providing a frozen sample including a plurality of whole cells and at least partially frozen liquid into a conduit;
moving the frozen sample in the conduit;
exposing the frozen sample to energy while the frozen sample is in the conduit to thaw the frozen sample and form a thawed sample;
exposing the thawed sample to focused acoustic energy while the thawed sample is in the conduit to create a focal zone of acoustic energy at the thawed sample to lyse the plurality of whole cells and release contents of the plurality of whole cells and/or to disassociate biomolecules from the plurality of whole cells; and
moving the thawed sample including the released contents of the plurality of whole cells in the conduit to a subsequent treatment area of the conduit.

28. The method of claim 27, wherein the step of moving the frozen sample in the conduit includes moving the frozen sample to a thawing zone of the conduit.

29. The method of claim 27, wherein the step of exposing the frozen sample to energy while the frozen sample is in the conduit to thaw the frozen sample includes exposing the frozen sample to focused acoustic energy to thaw the frozen sample.

30. The method of claim 27, wherein the step of exposing the frozen sample to energy while the frozen sample is in the conduit to thaw the frozen sample includes exposing the frozen sample to heat energy to thaw the frozen sample.

31. The method of claim 27, wherein the step of moving the thawed sample to a subsequent treatment area of the conduit includes moving the thawed sample into a separation chamber to separate protein material in the released contents from other material in the released contents.
